(19) United States
(12) Patent Application Publication
(10) Pub. No.: US 2019/0247847 A1
(43) Pub. Date: Aug. 15, 2019

(54) METHODS AND APPARATUS FOR PERFORMING METABOLIC MEASUREMENTS OF INDIVIDUAL CELL TYPES WITHIN NON-CONTACT CO-CULTURED SYSTEMS

(71) Applicant: Agilent Technologies, Inc., Santa Clara, CA (US)

(72) Inventors: Chong Wing Yung, San Jose, CA (US); Genevieve Van De Bittner, Campbell, CA (US); Smruti Madan Phadnis, San Jose, CA (US)

(21) Appl. No.: 15/896,255

(22) Filed: Feb. 14, 2018

(51) Int. Cl. B01L 3/00; C12M 1/32; C12M 1/00; C12M 1/12; C12M 1/34; G01N 21/64

(52) U.S. Cl. CPC B01L 3/5025; C12M 23/12; C12M 27/18; C12M 25/04; C12M 41/32; B01L 2300/0829; B01L 3/50853; G01N 21/6454; B01L 2300/046; B01L 2200/026; C12M 29/10

(57) ABSTRACT

The invention relates generally to methods and apparatus that measure one or more properties of an individual cell type in a non-contact co-culture. More specifically, the invention relates to a novel multiwell plate that allows separate but simultaneous metabolic measurements of cell populations in non-contact co culture.

### CROSS-REFERENCE TO RELATED APPLICATIONS

None.

### FIELD OF THE INVENTION

The invention relates generally to methods and apparatus that measure one or more properties of an individual cell type in a non-contact co-culture. More specifically, the invention relates to a novel multiwell plate that allows separate but simultaneous metabolic measurements of cell populations in non-contact co culture.

### BACKGROUND OF THE INVENTION

Multiwell plates are widely used for conducting separate measurements on cells in parallel and/or simultaneously and are commercially available in a variety of well formats from vendors such as Agilent Technologies, Sigma-Aldrich, Thomas Scientific, and others. Multiwell plates for tissue culture are available in 6, 12, 24, 48, 96, 384- and 1536-well formats, and coated and non-coated plates are available for adherent cell cultures and suspension cultures, respectively.

Multiwell plates are often used for conducting measurements on homogenous cell populations. However, pure homogenous cell populations rarely exist in nature. Heterogeneous cell populations are required for functionality, long-term survival and propagation.

Co-cultures are in vitro models that provide a physiologically relevant way of demonstrating in-vivo like tissue morphology and function. There are two kinds of co-culturing techniques that are used frequently. In a non-contact co-culture, cells are separated so as to prevent physical contact, but paracrine signaling is allowed. Paracrine signaling occurs in multicellular organisms by diffusion of one or more signaling molecules into extracellular space, or through long-range conduits or extracellular vesicles. Non-contact co-culture physically separates two or more cell populations, but allows signaling between them, such as by allowing liquid medium to pass from one cell population to another cell population. In a contact co-culture, different cell types are in physical contact, which permits juxtacrine signaling or contact-dependent signaling, involving cell-cell or cell-extracellular matrix signals that requires close contact. In contact co-culture, multiple cell types may be mixed together at a known or an unknown ratio.

Co-culturing equipment such as permeable supports have been used to study non-contact co-cultures. For example, Transwell® cell culture inserts (available from Corning Inc., Tewksbury, Mass.) are used by first adding medium and a first type of cell to the wells of multiwell plate, followed by adding the cell culture inserts to each well. Then, cell culture medium and a second type of cell are added to the inside compartment of the cell culture insert. The cell culture inserts, which have solid walls and a porous membrane bottom, hang on the tops of well, effectively forming upper and lower compartments within the well, and allow bi-directional exchange of media, reagents and sometimes migration of cells through the porous membrane into either sides of the compartments. The insert has a depth less than the depth of the well, so that the insert's membrane does not contact the well bottom, preventing co-cultured cell monolayers in the lower compartment from being damaged when the insert is in place. Non-contact co-culture equipment such as Transwell® inserts have been used to study cell populations in co-culture.

There is generally a need to characterize the activities and dynamics of each cell population in detail, as well as the inter-population interactions in a co-culture. Usually this entails the measurement of components of the cell culture medium such as released cytokines, growth factors, and others, as well as cellular mRNA and other RNA measurements, to assess the inter-population interactions in a co-culture. These are either end-point data collection or sampling during the course of the culture for off-line analysis (Goers L, 2014).

However, there is a need for assessing inter-population interactions in real time, and cell culture inserts may not easily interface with online measurement devices such as plate readers. Furthermore, such inserts are generally unsuitable for online monitoring of multiple and separate cell populations, especially simultaneously. The problem is further compounded by the fact that there is a lack of fast, simple, high-throughput assays to measure the bioenergetics or metabolic state of individual cell types simultaneously within the co-cultures (Schmidt J K, 2011).

Protein and aptamer-based sensors have been used to detect indicators of metabolic state such as glutathione/glutathione disulfide, NAD⁺/NADH, ATP/AMP, H₂O₂ or pH. (Roma L P, 2012; Sugiura K, 2015). These sensors are tagged with fluorophores and analyzed by confocal microscopy. This is not a scalable method and in most cases it is an endpoint assay.

U.S. Pat. No. 7,186,548 (Li) discloses a cell co-culture tool includes a body, an outer wall surrounding the body, and more than one vessel within the perimeter of the outer wall. Each vessel has a top edge below a rim of the outer wall.

U.S. Pat. No. 9,494,577 (McGarr et al.) discloses an apparatus including a plurality of wells for conducting analysis of three-dimensional cell samples (e.g., tissue samples) and methods for experimenting with a three-dimensional sample. A removable insert for use with the apparatus enables plunger-driven perfusion of the three-dimensional sample.

### SUMMARY OF THE INVENTION

These and other features and advantages of the present methods and apparatus will be apparent from the following detailed description, in conjunction with the appended claims.

### BRIEF DESCRIPTION OF THE DRAWINGS

FIG. 1A is a model of an embodiment of the present multiwell plate having interconnected wells.

FIG. 1B is a cut-away view of the same embodiment of the present multiwell plate.

FIG. 2 is a schematic cross sectional view of a well of a system for metabolic measurements comprising a multiwell plate, sensors and a barrier configured for forming a reduced volume.

FIG. 3 is a schematic illustration of a measurement system and apparatus in accordance with an embodiment of the invention.

The present teachings are best understood from the following detailed description when read with the accompanying drawing figures. The features are not necessarily drawn to scale. Wherever practical, like reference numerals refer to like features.

### DETAILED DESCRIPTION OF THE INVENTION

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

### Definitions

As used herein, the term “co-culture” refers to a culture or culturing of cells in the presence or influence of another population or type of cells, such as by growing or maintaining a population of cells in a manner where one or more signals are received from a second population or type of cells. For example, one cell type may serve as a stimulating cell, and another cell type may serve as the target cell which is stimulated.

As used herein, and in addition to their ordinary meanings, the terms “substantial” or “substantially” mean to within acceptable limits or degree to one having ordinary skill in the art.

As used herein, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same.

In the present disclosure, numeric ranges are inclusive of the numbers defining the range. It should be recognized that chemical structures and formula may be elongated or enlarged for illustrative purposes.

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference.

As used in the specification and appended claims, the terms “a,” “an,” and “the” include both singular and plural referents, unless the context clearly dictates otherwise. Thus, for example, “a moiety” includes one moiety and plural moieties.

Multiwell Plates with Interconnected Wells

As one aspect of the present invention, a multiwell plate is provided for a cell population in non-contact co-culture. The multiwell plate comprises a frame having a frame surface and frame sides extending from the frame surface. The multiwell plate also comprises a plurality of wells. Each well has an open end, a closed end opposite the open end, and at least one wall between the open end and the closed end. The open end of each of the wells is surrounded by the frame surface. Preferably, the wells extend from the frame surface in the same direction as the sides, and/or the walls of the wells do not extend above the frame surface. The multiwell plate also comprises at least one channel interconnecting at least two of the wells and configured to allow liquid medium to pass through the channel or between the wells interconnected by the channel. The channel is open to the frame surface and contiguous with the open ends of the two interconnected wells.

In some embodiments of the multiwell plate, at least two interconnected wells (preferably, each of the interconnected wells) has a depth d1, and the channel connecting those two wells (preferably, each of the channels of the multiwell plate) has a depth d2. In some embodiments, the difference between depth d1 and depth d2 is sufficient to prevent movement of cells between the two interconnected wells. Depth d1 will generally be greater than depth d2, as measured from the open ends of the two interconnected wells (or from a plane defined by the open ends of the wells of the multiwell plate). By way of example, depth d1 can be between 1 mm and 20 mm, and depth d2 can be between 0.2 mm and 17 mm, with the proviso that depth d1 is greater than depth d2. Alternatively, each well has a depth d1 between 2 mm and 10 mm, and/or each of the channels has a depth d2 between 0.5 mm and 5 mm. In some embodiments, the difference between d1 and d2 is less than 3 mm.

In some embodiments, at least one channel is configured to prevent cells from passing through the channel or between the wells interconnected by the channel, such as by its physical layout or by including additional elements. For example, the channel can be configured to receive a membrane which is permeable to liquid but impermeable to cells. In some embodiments, the membrane is selectively permeable based on one or more of molecular size, charger, hydrophilicity or hydrophobicity, or other criteria. In some embodiments, the membrane is substantially impermeable to proteins.

The walls defining the plurality of wells can be integral with the frame, and they may extend from the frame surface. The multiwell plate can have any number of wells, preferably 24, 48, 96, 384, or 1536 wells.

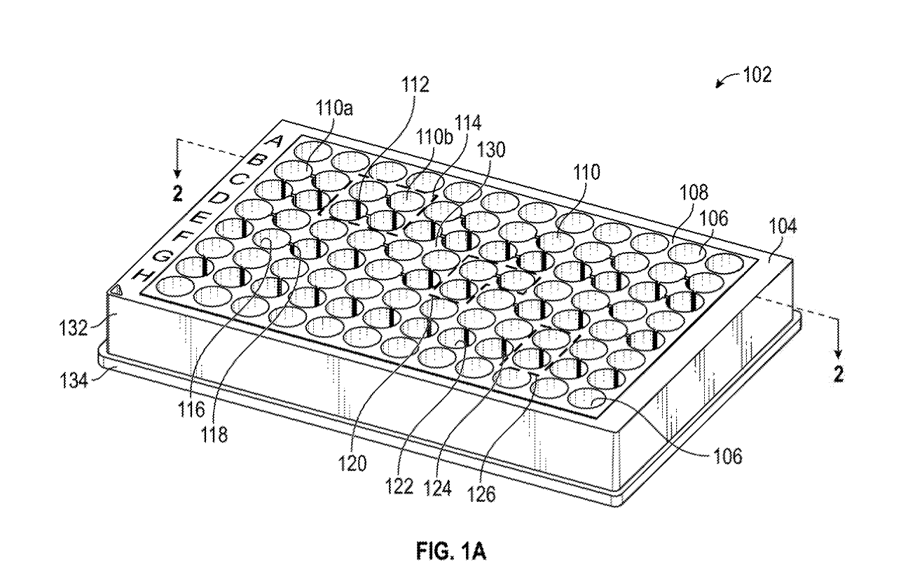

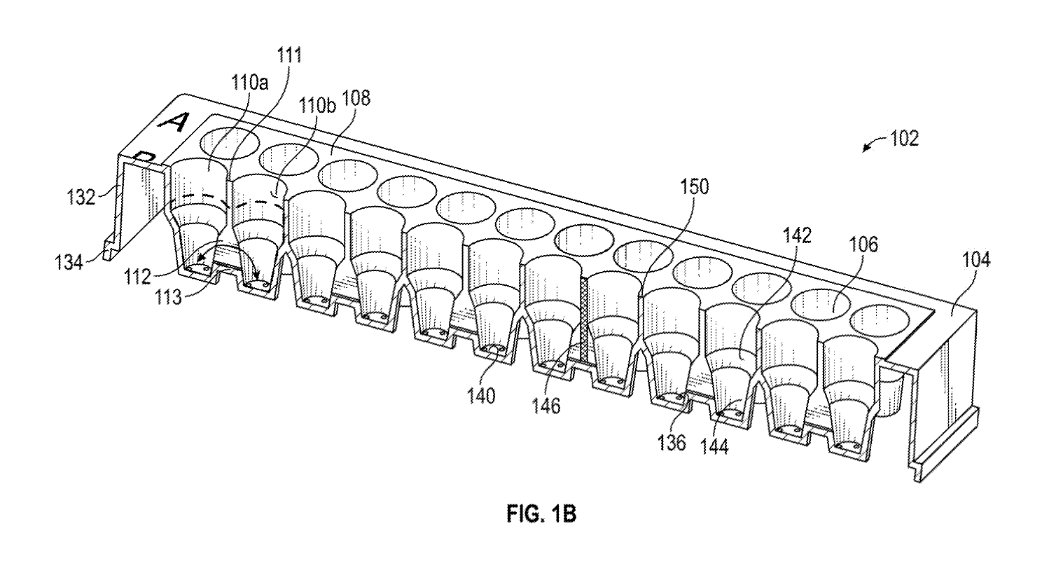

FIG. 1A is a 3D-model of an embodiment of the present multiwell plate. The multiwell plate can have any number of combinations of interconnected wells. By way of examples, FIG. 1 shows sets having 2, 3 and 4 interconnected wells, respectively, grouped in dashed boxes. More particularly, FIG. 1A shows an embodiment of a multiwell plate 102 comprising ninety-six wells and a frame 104. In this embodiment, the wells are defined by the frame 104, in that the walls and closed end of each well are integral with or part of the structure of the frame 104. FIG. 1A shows four types of wells. The wells 106 of the first and last rows (A and H) are standard wells which are isolated by walls 108 from any other well. Rows B and C comprise wells 110 which are interconnected with three other wells 110 by channels 112. A set 114 of four interconnected wells is demarcated by dashed lines, which may or may not be visibly indicated on the frame surface 130 of multiwell plate 102. Rows D and E comprise wells 116 interconnected with two other wells by channels 118. A set 120 is demarcated by dashed lines, which may or may not be visibly indicated on multiwell plate 102. Rows F and G comprise wells 122 interconnected with only one other well by a channel 124. A set 126 is demarcated by dashed lines, which may or may not be visibly indicated on multiwell plate 102. Multiwell plate 102 includes a frame surface 130 surrounding the open ends of the wells 106, 110, 116, 122, as well as frame sides 132 and a frame base 134. The open ends can be considered as defining a plane, and in some embodiments, the channels are open to that plane, and the channels do not have a wall or enclosure opposite their closed ends. In some embodiments, the walls 108 of the wells 110 do not extend past the frame surface 130. This facilitates use of the multiwell plate 102 in automated systems.

FIG. 1B is a cut-away view of the same embodiment of the present multiwell plate along the plane of the dash line in FIG. 1A. A liquid medium is shared amongst the interconnected wells via the interconnecting channel. More particularly, FIG. 1B shows a channel 112 that allows liquid to pass between first well 110a and second well 110b. The channel 112 can be the absence of a portion of a wall 108 that separates or isolates wells. Alternatively, the channel 112 is formed by a channel portion 111 and a channel closed end 113. The channel 112 is formed by a channel portion 111 of a wall 108 shared by two interconnected wells 110a, 110b, which can be a slot, hole, or other aperture in the wall separating the two wells. The channel closed end 113 has a lesser depth, as measured from the open ends than well closed end 140, and channel 112 is open in the direction of the open ends of the two interconnected wells. The channel 112 has a depth less than the depths of the two wells 110a, 110b that it interconnects. In some embodiments, channel 112 has a membrane 146 between the wells 110a, 110b, which separates the cells in each of the wells. Channel closed end 113 is higher than closed ends 140 of the wells 110a, 110b, relative to the base 134 of the multiwell plate. In some embodiments, well closed ends 140 can have barrier stops 136 to engage a barrier.

In some embodiments of the present methods and apparatus, each of the wells has a conical shape at least partially along a depth of the well, though the wells can be any desired shape (cylindrical, fully conical, rectangular, or other). FIG. 1B also illustrates that the wall of the wells can be conical, for example, with a first conical portion 142 and a second conical portion 144.

In some embodiments, the wells of the multiwell plate are coated, such as to promote or discourage cell adherence. Coatings of laminin, collagen, or lysine can be disposed on the wells of multiwell plates to promote adherence and can be used in multiwell plates adapted for measuring properties of adherent and non-adherent cells. In some embodiments, where use with for non-adherent cells is contemplated, the wells include one or more features to prevent cells from passing through the channel, such as coatings to discourage cell adherence, or a membrane disposed in the channel. In some embodiments, cells which are part of a tissue sample or three dimensional cell culture may not be adhered. In some embodiments, a tissue sample or three dimensional cell culture is adhered on a bottom of a well and/or on a coated surface so as to prevent movement.

It is contemplated that movement between wells is prevented if there is no movement or essentially no movement of cells between connected wells. In some embodiments, there might be a minimal movement of cells between connected wells during the mixing and/or measurement process or during the cell culture period. For example, mixing might dislodge some cells and make it possible for them to travel to neighboring wells. As another example, dead cells may become non-adherent and travel to neighboring wells during cell culture. Movement between wells is prevented so long as the number of cells that move between connected wells is expected to be small with essentially no impact on the cell measurements.

### Measurement of Co-Cultured Cells

The present methods and apparatus are especially useful in the field of micro-respirometry, which includes quantitatively measuring the bioenergetics or metabolic state of a small number of cells, as opposed to respirometry performed on whole animals. In the past, micro-respirometry was performed with microscopic glass flow cells that utilized milliliters of cell culture and Clark electrodes for measuring cell metabolism. This technique is not microscopic, facile or high-throughput. Flux analyzers and assays from Seahorse Bioscience provided improved technology for micro-respirometry by introducing comprehensive assays that can be easily performed in 8, 24 and 96 plastic cell culture plates. The resulting technology enabled complex characterization of both the glycolysis and oxidative phosphorylation pathways, by introducing various stimulants, inhibitors and custom drugs and measuring changes in oxygen consumption and proton production.

Cell phenotype is highly associated with the state of cell metabolism and the degree to which cells are reliant on oxidative phosphorylation or glycolysis. This association can be used to determine the function and/or state of cells (e.g., activation of immune cells, stem cell differentiation, iPCS reprogramming, oncogenesis) through characterizing the static and dynamic metabolic phenotype. While many experiments in micro-respirometry are done with mono-cultures of a single cell type, emerging questions in biology relate to the interaction of different cells types, which is required in multicellular organisms. For example, scientists seek to understand how, when and what other cells are activating immune cells, going beyond simply understanding whether immune cells are activated or not. Such questions can be answered with co-culture experiments, but at this point, there has not been a solution for performing micro-respirometry on co-cultures and attributing metabolic measurement to specific types or groups of cells. The present invention details the method and apparatus for measuring individual types or groups of cells even as they live or grow in co-culture.

### Systems For Metabolic Measurements of Cell Populations

As another aspect of the present invention, the present methods and apparatus are provided as a system for metabolic measurements of individual cell types in a non-contact co-culture. The system can include a multiwell plate as described herein. The system can also include one or more sensors configured for metabolic or other measurements of a cell population in a well of the multiwell plates.

In some embodiments, a system for metabolic measurements of a cell population containing individual cell types within non-contact co-cultures comprises a multiwell plate. The multiwell plate comprises a frame having a frame surface and frame sides extending from the frame surface. The multiwell plate also comprises a plurality of wells. Each well has an open end, a closed end opposite the open end, and at least one wall between the open end and the closed end. The open end of each of the wells is surrounded by the frame surface. The multiwell plate also comprises at least one channel interconnecting at least two of the wells and configured to allow liquid medium to pass through the channel or between the wells interconnected by the channel.

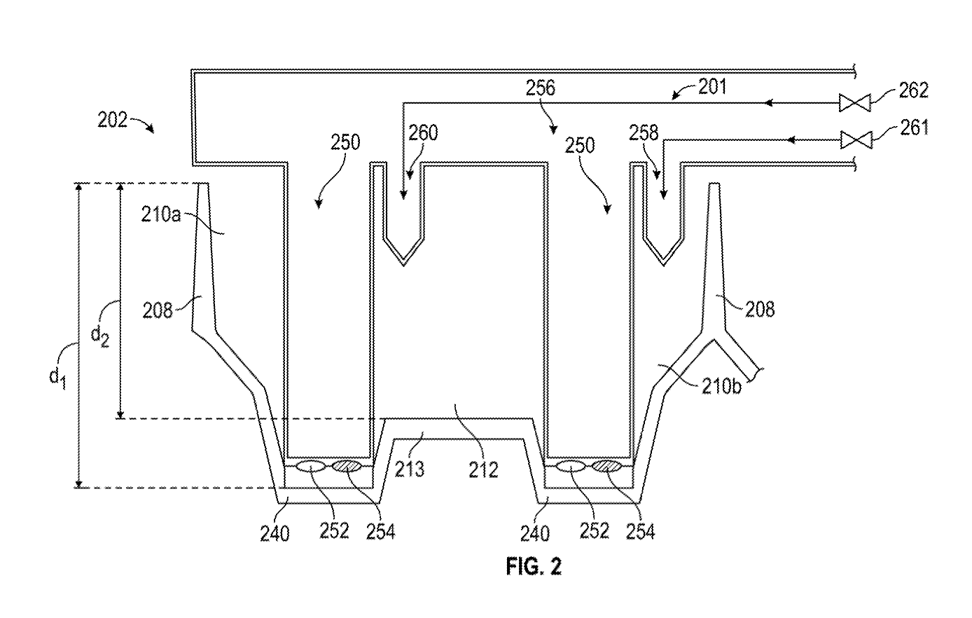

FIG. 2 is a schematic cross sectional view of a well of a multiwell plate having sensors and a barrier that forms a reduced volume and isolates the cells within a single well. The system 201 shown in FIG. 2 includes a multiwell plate 202 having a plurality of wells, two of which are shown. Wells 210a and 210b are interconnected by a channel 212 which allows liquid media to pass between those wells. Channel closed end 213 has a depth d2 less than the depth d1 of well closed end 240. The system 201 includes barriers 250 which are configured to be inserted into the wells 210a, 210b for mixing or measurement or other functions. Barriers 250 can be inserted and removed one or more times to mix the liquid medium. Sensors 252, 254 are disposed on an inserted end of the barriers 250. Barriers 250 and the closed ends 240 of the wells form a reduced volume or microchamber where metabolic measurements of individual cell types are made. Barriers 250 prevent the liquid medium from being exchanged between the different cell types during measurement. FIG. 2 depicts a schematic cross section of a well 110 of an embodiment of the invention showing a cartridge 256 having barrier 250 and ports 258, 260 used to add molecules to wells so as to alter the microenvironment of the cell samples under examination. Details of a barrier system of the existing, commercially available Seahorse XF Analyzer are described in US 20080014571, the disclosure of which is incorporated herein by reference. The cartridge 256 may deliver gases, e.g., an external gas 262, i.e., from a gas cylinder, or liquids 264 to the wells.

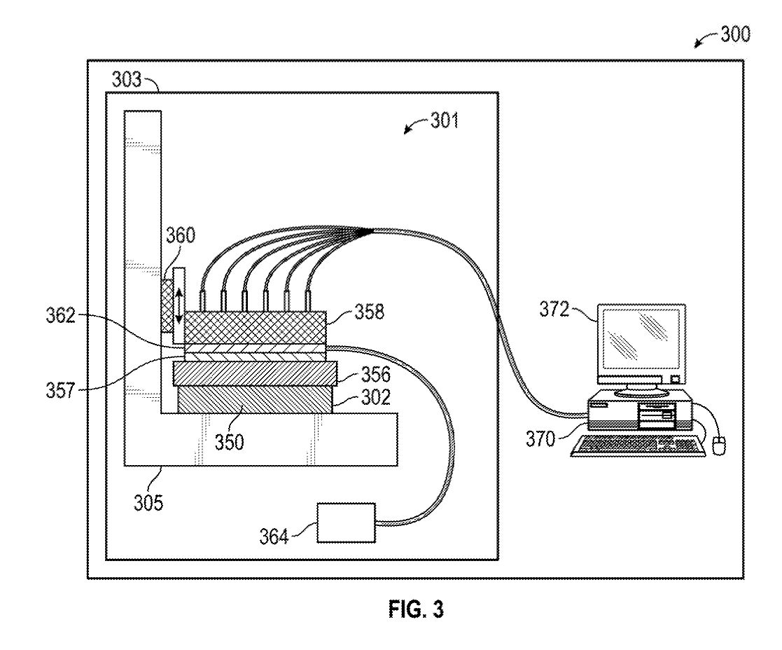

FIG. 3 is a schematic illustration of an embodiment of the present measurement methods and apparatus. The apparatus 300 includes a measurement system 301 disposed in a housing 303 and includes a cartridge 356 having or configured to receive a plurality of barriers or other sensor structures and a plurality of fluid ports, and a stage 305 adapted to receive the present multiwell plate 302 as described herein. Stage 305 can be configured to securely hold or attach to a base 134 of a multiwell plate. Stage 305 can also contain one or more signal detectors (such as images) to detect a signal from one or more sensors 252, 254. The cartridge 356 is disposed above, and adapted to mate with, the multiwell plate 302. The cartridge 356 optionally is held by a cartridge holder 357 adapted to receive the cartridge 353. The apparatus also includes a mounting block 358, which can reciprocate as shown by the double headed arrow, preferably powered by a motor (not shown), including an elevator mechanism 360. The elevator mechanism 360 may be adapted to move the cartridge 356 relative to the stage 620, or well plate 400. The mounting block includes a multiplexer 362 attached to a supply or reservoir 364. The supply 364 is in fluid communication with the cartridge, and is used to impel the delivery of fluid from a port in the cartridge 356 to a well in the multiwell plate 302. A plurality of barriers 350 with sensors are adapted for insertion into the plurality of wells in multiwell plate 302, and may be used to gather data indicative of the state of cells disposed in wells in the multiwell plate 302.

The present methods and apparatus may include an automated electro-optical measurement system. The apparatus may also include a computer, with the automated electro-optical measurement system being in electrical communication with the computer.

The measurement system 301 is controlled by a controller 370, that may be integrated with a computer 372, that may control the elevator mechanism 360, the sensors, and/or other elements of the system 301.

The apparatus described herein is a modification of the apparatus disclosed in U.S. Pat. Nos. 9,170,253 and 9,494,577 (which are incorporated by reference herein), and enables experimentation with and analysis of cells in non-contact co-culture. Further teachings about metabolic measurements in reduced volumes can be found in U.S. Pat. Nos. 8,697,431 and 9,170,253 (Teich et al.), which disclose a method of analyzing cells disposed in media within a vessel includes the steps of providing a vessel having an original volume of media about the cells, reducing the original volume of media about at least a portion of the cells to define a reduced volume of media, and analyzing a constituent related to the cells within the reduced volume of media. An apparatus for analyzing cells includes a stage adapted to receive a vessel holding cells and a volume of media, a plunger adapted to receive a barrier to create a reduced volume of media within the vessel including at least a portion of the cells, the barrier adapted for insertion into the vessel by relative movement of the stage and the plunger, and a sensor in sensing communication with the reduced volume of media, wherein the sensor is configured to analyze a constituent disposed within the reduced volume.

### Sensors for Analyzing Cell Populations

In the present methods and apparatus, one or more sensors can be used to measure physiological properties of a cell population in non-contact co-culture. The sensors can be a fluorescent sensor, a luminescent sensor, an ISFET sensor, a surface plasmon resonance sensor, a sensor based on an optical diffraction principle, a sensor based on a principle of Wood's anomaly, an acoustic sensor, or a microwave sensor.

In some embodiments, at least one sensor is an optical sensor such as a fluorescent or chromogenic molecule. Preferred optical sensors for are fluorophores. When oxygen is an analyte to be sensed, preferred sensors include porphyrin or rhodamine compounds, which can be embedded in an oxygen permeable polymer, e.g., silicone rubber. When H⁺ is an analyte to be sensed, preferred sensors include fluorescein compounds, whose signal decreases upon protonation of the dye, and which can be embedded in a carrier polymer or covalently attached to a hydrophilic polymer.

In some embodiments, at least one sensor is a capture reagent such as an antibody or antigen-binding fragment thereof. The nature of the sensor generally does not form an aspect of embodiments of this invention.

The one or more sensors are adapted to sense one or more analytes of the sample or the liquid media in contact with the sample. The analyte can be a dissolved gas (e.g., O₂, CO₂, NH₃, NO₂), an ion (e.g., H⁺, Na⁺, K⁺, Ca⁺⁺), a protein (e.g., cytokines, insulin, chemokines, hormones, antibodies), a substrate (e.g., glucose, a fatty acid, an amino acid, glutamine, glycogen, pyruvate), a salt, a mineral, and/or a reactive oxygen species (e.g. H₂O₂, O₂⁻, OH). The analyte may be extracted from the media by at least a portion of the cells. The analyte may be secreted into the media by at least a portion of the cells. In some embodiments, a first analyte is sensed for a cell population to perform a metabolic measurement. The first analyte can be H⁺ and the metabolic measurement is ECAR. In some embodiments, a second analyte is sensed simultaneously with the first analyte. For example, the second analyte can be O₂ and the metabolic measurement can be OCR.

Sensing an analyte may include sensing the presence and/or the concentration of the analyte. Sensing the analyte may include sensing a first concentration of a first analyte, sensing a second concentration of a second analyte, and determining a relationship between the first concentration and the second concentration. Sensing the analyte may include sensing a rate of change of a concentration of the analyte. Sensing the analyte may include determining a parameter such as cell viability, cell number, cell growth rate, response to at least one of a drug, a toxin or a chemical, detection of an entity, and internalization.

### Cell Populations for Non-Contact Co-Culture

The cell populations used in the present methods and apparatus may include bacteria, fungus, yeast, a prokaryotic cell, a eukaryotic cell, an animal cell, a human cell, and/or an immortal cell. At least a portion of the cells may be attached to a surface of the vessel. At least a portion of the cells may be suspended in the media. At least a portion of the cells may include living tissue. In some embodiments, at least a portion of cells of the first and second cell populations are adhered to a closed end or a wall of the wells.

The present methods and apparatus can be used to perform individual metabolic measurements on two cell types in non-contact co-culture, where at least one of the cell types influences growth or function of the other cell type. For example, the co-culture can include one or more types of immune cells, such as macrophages and fibroblasts. Implantation of any foreign material into living tissue evokes a host inflammatory response described as foreign body response (FBR). Macrophages and fibroblasts are primary FBR effector cells acting in concert in local implant associated inflammation, cell recruitment, implant degradation, fibrosis, and chronic unresolved healing. Macrophages and fibroblasts in the FBR communicate via soluble autocrine and paracrine signals as well as juxtacrine signals associated with direct cell-cell contacts. Pro-inflammatory cytokines are secreted by both macrophages and fibroblasts are immediately upregulated post-injury and remain upregulated in the presence of a foreign material.

Other cell types that may be used in the present methods and apparatus include liver cells, heart cells, kidney cells, spleen cells, neurons, epithelial cells, thyroidal cells, adrenal cells, iris cells, cancer cells.

The present methods and apparatus can be used to perform individual metabolic measurements on two cell types in co-culture having differences in rates of glycolysis. For example, the Warburg effect arises because most cancer cells predominantly produce energy by a high rate of glycolysis followed by lactic acid fermentation in the cytosol. Most normal cells exhibit a comparatively low rate of glycolysis followed by oxidation of pyruvate in mitochondria. A hallmark of malignant cancers is an elevated glucose uptake even under normal oxygen conditions, known as “aerobic glycolysis” or the Warburg effect. Cells exhibiting a Warburg effect catabolize glucose at a high rate.

The present methods and apparatus enable individual metabolic measurements on cells in co-culture wherein the Warburg effect is present between the first cell type and the second cell type. As a first cell type, an established cancer cell lines such as HeLA or PANC1 is used. As a second cell type, normal primary fibroblast cells such as BJ or WS1 from ATCC is used. A first cell population having cells of the first cell type is placed in a first well of a multiwell plate. A second cell population having cells of the second cell type is placed in a second well of the multiwell plate, wherein the first and second wells are interconnected by a channel. The first and second cell populations are grown in the same medium but physically separated in the interconnected wells. The influence of the normal cells on the metabolism of the cancer cells is determined by metabolic measurements on the cancer cells, or the influence of the cancer cells on the metabolism of the normal cells is determined by metabolic measurements on the normal cells.

Known cell lines can be used as a cell type in the present methods and apparatus, for example, in assays comparing the known cell line to a cell population obtained from a test subject. Examples of known cell lines include, but are not limited to, C8161, CCRF-CEM, MOLT, mIMCD-3, NHDF, HeLa, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, H-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). These or other cell lines can be employed as the first cell type or the second cell type in the present methods and apparatus. In some embodiments, the first cell type is in a cell population taken from a subject (such as a human patient), and the second cell type is a known cell line.

### Methods for Performing Metabolic Measurements

As another aspect of the present invention, a method is provided for performing metabolic measurements on a cell population in non-contact co-culture. The method comprises the steps of placing a first cell population in a first well of a multiwell plate and placing a study material, such as a second cell population, in a second well of the multiwell plate. The multiwell plate comprises at least one channel that interconnects the first well and the second well so as to allow liquid to pass. The method also comprises the step of allowing liquid medium to move between the first and second well without movement of cells of the first cell population and the study material between the first and second wells. The method also comprises one or both steps of sensing one or more analytes and/or performing one or more metabolic measurements of the first cell population.

In some embodiments, the liquid medium is allowed to move for a co-culture period (for example, at least an hour, or at least a day), and the first cell population is measured after the co-culture period is ended. In some embodiments, movement of liquid medium to or from the first cell population is prevented, such as by moving a barrier into the first well, before or during the measuring of the first cell population. The methods can also include the steps of forming a reduced volume after the co-culture period and before or during the measuring of the first cell population, such as by moving a barrier into the first well at a depth between the channel and the closed end of the first well. Thereafter, the barrier can be withdrawn to increase the reduced volume of liquid medium about the cells and/or allow liquid medium to pass between interconnected wells.

In some embodiments, a method of performing metabolic measurement of a cell population in a non-contact co-culture comprises providing a multiwell plate according to any of the embodiments described herein. A first cell population containing one or multiple cell types or tissue of interest is placed in a first well of the multiwell device. A second cell population containing a one or multiple cell types or tissue of interest is placed in a second well of the multiwell device. Liquid is allowed to pass between the first and second wells for a period. After the period, the first well is isolated from the second well so that liquid does not pass between them. Then one or more metabolic measurements are performed for the first cell population, and optionally for the second cell population. The metabolic measurements can include sensing one or more analytes. In some embodiments, the first cell population comprises at least one cell type different than the second cell population. In some embodiments, the first cell population and the second cell population contain the same cell type(s).

In some embodiments, the method employs a study material other than (or in addition to) a second cell population. The study material can comprise organic and inorganic materials (for example, pieces of metal or plastic, paint chips), viruses, prions, nucleic acids, vesicles, artificial cells, antigens, medications, pharmaceutical formulations with controlled released properties, or other materials.

The present method and apparatus can be applied in a variety of fields, including biological research, drug discovery, and clinical diagnostics. For example, as a drug discovery tool, the device can be used to screen various molecules for an effect on cellular metabolism in co-culture, protein secretion, or intra/extra cellular ion exchange. The device can also be used to determine the health of cells in co-culture both before and after a conventional assay is performed, thereby improving the performance of such an assay.

The present methods apparatus for performing metabolic measurements on individual cell types in co-culture makes use of the new multiwell plates that include interconnected wells. In the present multiwell plate, at least one set of 2, 3, 4 or X wells are interconnected with channels such that cell culture media is shared amongst the interconnected wells as illustrated in FIG. 1A. X can be any integer between 2 and the total number of wells in the plate (for example, 8, 12, 24, 48, 96 or more). Alternatively, X is 2, 3, 4, 5 or 6. A multiwell plate can have one or more sets of interconnected wells in the well plate, such as 2, 3, 4 or Y sets. Y can be any integer between 1 and half the total number of wells in the plate (for example, 4, 6, 12, 24, 48 or more). In some embodiments, where X¹ is the number of interconnected wells in a set, and Y¹ is the number of sets in the multiwell plate, X¹ is 2 and Y¹ is 12, 24, 36, 42, or 48, alternatively X¹ is 3 and Y¹ is 8, 24, 28 or 32, alternatively X¹ is 4 and Y¹ is 18, 21 or 24, alternatively, a combination of the foregoing sets. In some embodiments, the multiwell plate has sets with a different number (X²) of wells within a number (Y²) of alternative sets, and X² and Y² can be any of the numbers set forth herein for X¹ and Y¹, respectively. In some embodiments, the multiwell plate includes wells that are isolated from (or not interconnected with) any other well, which may be desirable for use with a control.

Liquid media shared by interconnected wells can thus convey molecular signals (e.g. hormones, cytokines, metabolites, catabolic precursors, waste products etc.) between the wells, which are seeded with X number of types or groups (mixture) of cells. For example, with two interconnected wells, “A” cell type can be grown in well-1 and “B” cell type can be grown in well-2.

In some embodiments, a cartridge holding one or more sensors and/or one or more barriers is used to conduct metabolic measurements of cell samples in the present well plate. For example, a Seahorse cartridge can be used. While the Seahorse cartridge is raised, liquid medium is shared amongst well 210a and well 210b and “A” and “B” cells can communicate or signal to one another (FIG. 2). During the measurement phase, the Seahorse cartridge (barrier 250) is lowered towards the closed end of the well to create a semi-sealed transient microchamber. This action thus isolates the individual microchambers such that “A” and “B” cells are measured individually, even though the cells were previously co-cultured. Once the measurement phase is complete, the Seahorse cartridge (barrier 250) is raised or even repeatedly moved up-down to mix the shared media amongst the wells, continuing the co-culture conditions until the next measurement. The different combinations of co-cultures can be extrapolated to the number of interconnected wells, such that with 3 interconnected wells, one can co-culture A-B-A, B-A-B, A-B-C cell types, or with 4 interconnected wells, one can co-culture, A-B-A-B, A-A-A-B, B-B-B-A or A-B-C-D cell types, etc.

The present well plate can be used in a system with existing probe cartridge lids, instruments and assay reagents. Alternatively, cartridges may be specially designed for use with the present devices.

Interactions amongst different combination of cell types can be determined by comparing individual cell types in mono-cultures vs. cocultured environments, with or without various chemicals or drugs that can be introduced in the existing drug ports of the Seahorse probe cartridge. The present interconnected well plate is also compatible with further in situ downstream analyses, such as immunostaining with microscopy, cell viability, apoptosis, ATP, ELISA, etc.

In some embodiments, the present methods and apparatus include additional elements to promote or ensure liquid media mixing between adjacent wells of a cell culture plate. These elements can be employed to assess and understand the mixing efficiency imparted under various conditions, including 1) mixing of media between interconnected wells by simple diffusion through a channel, which will occur during cell culture, and 2) mixing of molecules between interconnected well introduced during experimentation or measurement, such as when a molecule is added into wells during an assay. Mixing can be assessed prior to, during, or after mixing provided by the lifting and lowering of a sensor cartridge. By understanding the mixing of molecules within a non-contact co-culture system, one may be able to design or modify assays with precise control over the presence of an added molecule across the interconnected wells. Furthermore, the present methods and apparatus can include a system for monitoring mixing between interconnected wells, in real-time during a cellular assay, which wells of an interconnected well system are exposed to the introduced molecule and on what timescale. In some embodiments, the introduced molecule is metabolism modulator, small molecule drug, beads, proteins, antibodies, or other.

For example, to monitor the mixing of the system, a marker such as a soluble fluorescent or chromogenic molecule can be utilized, which can be detected using the current optics of the Seahorse system or through modification of the Seahorse optical system. In some embodiments, these markers are attached to antibodies, proteins, beads etc., so that the movement and mixing measurements would most closely replicate the molecule that is being introduced to the wells during the assay, such as through the injection ports of the Seahorse system.

The present methods and apparatus provided numerous advantages over existing techniques for measuring properties of cells in co-culture. One does not have to de-convolute the signals from the different cell types because they are maintained in different wells, different cell types can be momentarily isolated for their individual measurement, and the measurements can be completed in a high-throughput format with multiwell plates.

### Exemplary Embodiments

Embodiment 1. A multiwell plate for a cell population in a liquid medium, the multiwell plate comprising a frame having a frame surface and frame sides extending from the frame surface; a plurality of wells, each well having an open end, a closed end opposite the open end, and at least one wall between the open end and the closed end; wherein the open end of each of the wells is surrounded by the frame surface; and at least one channel interconnecting at least two of the wells and configured to allow liquid medium to pass, and the at least one channel is open to the frame surface and contiguous with the open ends of the two interconnected wells.

Embodiment 2. The multiwell plate of embodiment 1, wherein a depth d1 of the at least two interconnected wells is greater than a depth d2 of the at least one channel interconnecting the wells.

Embodiment 3. The multiwell plate of embodiment 2, wherein the difference between depth d1 and depth d2 is sufficient to prevent movement of cells between the two interconnected wells.

Embodiment 4. The multiwell plate of embodiment 2, wherein each of the at least two interconnected wells has a depth d1 between 1 mm and 20 mm, and each of the at least one channel has a depth d2 between 0.2 mm and 17 mm.

Embodiment 5. The multiwell plate of embodiment 1, wherein the at least one channel is configured to prevent cells from passing.

Embodiment 6. The multiwell plate of embodiment 1, further comprising a membrane in the at least one channel, the membrane being permeable to liquid but impermeable to cells.

Embodiment 7. The multiwell plate of embodiment 1, wherein the channel is formed by a channel portion of a wall shared by two interconnected wells.

Embodiment 8. The multiwell plate of embodiment 1, wherein at least one wall of all of the plurality of wells are integral with the frame.

Embodiment 9. The multiwell plate of embodiment 1, wherein the multiwell plate comprises 8, 24, 48, 96, 384, or 1536 wells.

Embodiment 10. The multiwell plate of embodiment 1, wherein each of the wells have a conical shape at least partially along a depth of the wells.

Embodiment 11. A system for metabolic measurements of a cell population containing individual cell types within non-contact co-cultures, the system comprising a multiwell plate and one or more sensors configured for sensing one or more analytes in the wells. Any of the multiwell plates of the foregoing embodiments can be used.

Embodiment 12. The system of embodiment 11, further comprising barriers configured for insertion into the wells through the open ends so as to form a reduced volume subchamber within the wells.

Embodiment 13. The system of embodiment 11, wherein the one or more sensors are disposed on (such as by being embedded in) the barriers or in the closed ends of the well, for example in a permeable medium.

Embodiment 14. The system of embodiment 11, wherein the sensor is a capture reagent.

Embodiment 15. The system of embodiment 11, further comprising a signal detector in a position to detect a signal from the sensor.

Embodiment 16. The system of embodiment 15, further comprising a processor in communication with the signal detector.

Embodiment 17. A method for performing metabolic measurements on a cell population in non-contact co-culture, the method comprising the steps of: placing a first cell population in a first well of a multiwell plate; placing a study material (for example, a second cell population) in a second well of the multiwell plate, wherein the multiwell plate comprises at least one channel that interconnects the first well and the second well so as to allow liquid to pass; allowing liquid medium to move between the first and second well without movement of cells of the first cell population and the study material between the first and second wells; and sensing one or more analytes and/or performing one or more metabolic measurements of the first cell population.

Embodiment 18. The method of embodiment 17, wherein the liquid medium is allowed to move for a co-culture period (for example, at least an hour, or at least a day), and the first cell population is measured after the co-culture period is ended.

Embodiment 19. The method of embodiment 17, further comprising preventing movement of liquid medium to or from the first cell population, such as by moving a barrier into the first well, before or during the measuring of the first cell population.

Embodiment 20. The method of embodiment 17, further comprising forming a reduced-volume after the co-culture period and before or during the measuring of the first cell population, such as by moving a barrier into the first well at a depth between the channel and the closed end of the first well.

Embodiment 21. The method of embodiment 20, further comprising withdrawing the barrier to increase the reduced volume of media about the cells and allow liquid media to pass between interconnected wells.

Embodiment 22. The method of embodiment 17, wherein the cells comprise a cell selected from the group consisting of bacteria, fungus, yeast, a prokaryotic cell, a eukaryotic cell, an animal cell, a human cell, and an immortal cell.

Embodiment 23. The method of embodiment 17, wherein at least a portion of cells of the first and second cell populations are adhered to a closed end or a wall of the wells. In some embodiments, cells which are part of a tissue sample or three dimensional cell culture may not be adhered. In some embodiments, a tissue sample or three dimensional cell culture is adhered on a bottom of a well and/or on a coated surface so as to prevent movement.

Embodiment 24. The method of embodiment 17, further comprising sensing a first analyte of the first cell population, wherein the first analyte is H⁺.

Embodiment 25. The method of embodiment 24, further comprising sensing a second analyte simultaneously with measuring the first analyte, and the second analyte is O₂.

Embodiment 26. The method of embodiment 17, further comprising monitoring mixing between the first well and the second well.

Embodiment 27. The method of embodiment 17, further comprising adding a marker to the liquid medium, such as a soluble fluorescent or chromogenic molecule.

Embodiment 28. The method of embodiment 17, wherein the marker is attached to an antibody, a protein, small molecule, or a bead.

While the present disclosure applies to X combinations of cells types, the principle of operation can be proven by the following two-cell model systems, with cell types “A” and “B”.

### EXAMPLE 1

In this example, two distinct cell types are co-cultured in interconnected wells of a multiwell plate as described herein. The two cell types predominantly show differences in rate of glycolysis due to the Warburg effect. As a first cell type, an established cancer cell line such as HeLA or PANC1 is used. As a second cell type, normal primary fibroblast cells such as BJ or WS1 from ATCC are used. A first cell population having cells of the first cell type is placed in a first well of a multiwell plate. A second cell population having cells of the second cell type is placed in a second well of the multiwell plate, wherein the first and second wells are interconnected by a channel. The first and second cell populations are grown in the same liquid medium but physically separated in the interconnected wells.

After culturing the cells for 24 hours, a Seahorse glycolysis kit is used to assess the ECAR and/or OCR of the first cell type, the second cell type, or both. The influence of the normal cells on the metabolism of the cancer cells is determined by measuring ECAR and/or OCR of the cancer cells, or the influence of the cancer cells on the metabolism of the normal cells is determined by measuring ECAR and/or OCR of the normal cells.

### EXAMPLE 2

In this example, two distinct cell types are co-cultured in interconnected wells of a multiwell plate as described herein. The two cell types are human macrophages and fibroblasts. The macrophages and fibroblasts in the non-contact co-culture as allowed to communicate via soluble autocrine and paracrine signals in shared liquid medium, however juxtacrine signals associated with direct cell-cell contacts are not permitted. Thus, the multiwell plate allows for metabolic measurements on an immune cell population influenced by autocrine and paracrine signals, and isolated from juxtacrine signals. In this example, “A” cell type are macrophages and “B” cell type are fibroblasts cultured in the same liquid medium and physically separated in the interconnected wells.

After culturing the cells for 24 hours, a Seahorse glycolysis kit is used to assess the ECAR/OCR of the first cell type, the second cell type, or both.

As an independent assessment of the co-culturing effect, the conditioned liquid media is sampled, and an ELISA is performed to measure specific cytokines released by the macrophages and fibroblasts.

In view of this disclosure it is noted that the methods and apparatus can be implemented in keeping with the present teachings. Further, the various components, materials, structures and parameters are included by way of illustration and example only and not in any limiting sense. In view of this disclosure, the present teachings can be implemented in other applications and components, materials, structures and equipment to implement these applications can be determined, while remaining within the scope of the appended claims.

We claim:

1. A multiwell plate for a cell population in a liquid medium, the multiwell plate comprising:
a frame having a frame surface and frame sides extending from the frame surface;
a plurality of wells, each well having an open end, a closed end opposite the open end, and at least one wall between the open end and the closed end; wherein the open end of each of the wells is surrounded by the frame surface;
at least one channel interconnecting at least two of the wells and configured to allow liquid medium to pass, and the at least one channel is open to the frame surface and contiguous with the open ends of the two interconnected wells.

2. The multiwell plate of claim 1, wherein a depth d1 of the at least two interconnected wells is greater than a depth d2 of the at least one channel interconnecting the wells.

3. The multiwell plate of claim 2, wherein the difference between depth d1 and depth d2 is sufficient to prevent movement of cells between the two interconnected wells.

4. The multiwell plate of claim 1, wherein the at least one channel is configured to prevent cells from passing.

5. The multiwell plate of claim 1, further comprising a membrane in the at least one channel, the membrane being permeable to liquid but impermeable to cells.

6. The multiwell plate of claim 1, wherein the multiwell plate comprises 8, 24, 48, 96, 384, or 1536 wells.

7. The multiwell plate of claim 1, wherein each of the wells have a conical shape at least partially along a depth of the wells.

8. A system for metabolic measurements of a cell population containing individual cell types within non-contact co-cultures, the system comprising:
a multiwell plate comprising:
a frame having a frame surface and frame sides extending from the frame surface;
a plurality of wells, each well having an open end, a closed end opposite the open end, and at least one wall between the open end and the closed end; wherein the open end of each of the wells is surrounded by the frame surface;
at least one channel interconnecting at least two of the wells and configured to allow liquid medium to pass, and at least one channel is open to the frame surface and contiguous with the open ends of the two interconnected wells; and
one or more sensors configured for sensing one or more analytes in the wells.

9. The system of claim 8, further comprising barriers configured for insertion into the wells through the open ends so as to form a reduced volume subchamber within the wells.

10. The system of claim 8, wherein one or more sensors are disposed on the barriers or in the closed ends of the well.

11. The system of claim 8, wherein the sensor is a capture reagent.

12. The system of claim 8, further comprising a signal detector in a position to detect a signal from the sensor.

13. The system of claim 12, further comprising a processor in communication with the signal detector.

14. A method for performing metabolic measurements on a cell population in non-contact co-culture, the method comprising the steps of:
placing a first cell population in a first well of a multiwell plate;
placing a second cell population in a second well of the multiwell plate, wherein the multiwell plate comprises at least one channel that interconnects the first well and the second well so as to allow liquid to pass;
allowing liquid medium to move between the first and second well without movement of cells of the first and second cell populations between the first and second wells;
sensing one or more analytes and/or performing one or more metabolic measurements of the first cell population.

15. The method of claim 14, wherein the liquid medium is allowed to move for a co-culture period, and the first cell population is measured after the co-culture period is ended.

16. The method of claim 14, further comprising preventing movement of liquid medium to or from the first cell population, before or during the measuring of the first cell population.

17. The method of claim 14, further comprising forming a reduced volume after the co-culture period and before or during the measuring of the first cell population.

18. The method of claim 14, further comprising monitoring mixing between the first well and the second well.

19. The method of claim 14, further comprising adding a marker to the liquid medium.

20. The method of claim 14, wherein at least a portion of cells of the first and second cell populations are adhered to a closed end or a wall of the wells.
